(19) United States
(12) Patent Application Publication
(10) Pub. No.: US 2005/0075301 A1
(43) Pub. Date: Apr. 7, 2005

(54) Pharmaceutical preparation with RNA as hemostasis cofactor

(76) Inventors: Klaus Preissner, Giessen (DE); Fumie Nakazawa, Tokyo (JP); Christian Kannemeier, San Diego, CA (US); Juergen Roemisch, Voesendorf (AT)

Correspondence Address: Finnegan, Henderson, Farabow,;Garrett & Dunner, L.L.P., 1300 I Street, N.W., Washington, DC, 20005-3315, US

(21) Appl. No.: 10/631,896

(22) Filed: Aug. 1, 2003

(30) Foreign Application Priority Data
Aug. 6, 2002 (DE) 10236038.3
Mar. 3, 2003 (DE) 10309368.0

(51) Int. Cl. A61K 48/00; A61K 38/48

(52) U.S. Cl. 514/44; 514/7; 424/94.64

(57) ABSTRACT

Pharmaceutical preparations which comprise RNA or one or more coagulation-promoting fragments of RNA in amounts sufficient to promote coagulation and to promote fibrinolysis are described. They are in particular advantageously employed together with an activator for hemostatic processes such as factor VII-activating protease (FSAP)

The invention relates to pharmaceutical preparations which, besides coagulation factors, additionally comprise natural or synthetic ribonucleic acid (RNA) or bioactive fragments of RNA or RNA-degrading substances such as ribonucleases.

It is known that the processes of hemostasis (coagulation and fibrinolysis), which proceed locally and for a limited time after commencement of a vessel lesion, are controlled by specific interactions between activated vessel wall cells, platelets and protein factors. A localization of the proteins active in hemostasis on in particular negatively charged biomembranes at the site of injury is achieved via in most cases ionic interactions of the calcium-binding and vitamin K-dependent coagulation factors. These interactions, and thus the activation of the coagulation system and the extent of the formation of a fibrin clot, can be prevented in vitro by complexing substances such as ethylenediaminetetraacetic acid (EDTA) or citrate. The use of such anticoagulants is impermissible in vivo; here it is possible to prevent, by therapy with oral vitamin K antagonists, the calcium binding of the coagulation factors, so that the extent of hemostasis is reduced.

Besides the formation of negatively charged phospholipids through exposure of cell membranes of the platelets and other vascular cells, injury to the cell wall is also followed by exposure of intracellular components, especially of cytosolic proteins. It is a well-researched fact that the coagulation system comprises two different cascade-like activation pathways of the coagulation factors present in plasma. Depending on the inducing mechanism, the intrinsic or extrinsic pathway is preferred for initiating coagulation. When there is a tissue injury, thromboplastin (tissue factor, TF) is exposed with phospholipids of the affected cells as initiator of the extrinsic coagulation pathway. The particular function of coagulation factor VII in these processes is disclosed in German Offenlegungsschrift 199 03 693. The membrane-associated thromboplastin is able to bind both coagulation factor VII (FVII) and the circulating activated factor FVII (FVIIa). This TF-FVIIa complex leads, in the presence of calcium ions and lipids, to the binding of FX which is converted by limited proteolysis into its activated form (FXa). FXa in turn leads to activation of prothrombin to thrombin for fibrin formation and thus eventually wound closure.

According to current knowledge, further activation of the FVII bound to thromboplastin takes place in particular autocatalytically, but this is supported after initiation of the coagulation cascade in particular by FXa and thrombin, leading to a marked enhancement of the response cascade.

It was possible to infer from these findings that, in certain clinical situations, administration of FVIIa or FVIIa-containing concentrates is indicated. In patients suffering for example from hemophilia A who have developed, as a result of administration of FVIII, antibodies against FVIII, use is made of the so-called FVIII inhibitor-bypassing activity (FEIBA) of FVIIa. It has emerged in this connection that FVIIa is well tolerated and does not lead to a tendency to thrombosis, but is suitable for ensuring coagulation to a limited but sufficient extent. Recombinant FVIIa is already in use for therapy and prophylaxis. FVII obtained from blood plasma can likewise be activated and then used. According to a current knowledge, it is possible to use for this activation proteases such as thrombin, but these may as such themselves strongly activate coagulation and lead to a risk of thrombosis. It is therefore necessary for thrombin to be subsequently removed or inactivated, leading to losses of yield. The use of FXa or FIIa (thrombin) is frequently contraindicated because of the risk of thrombosis associated therewith and is indicated only in emergency cases, for example in cases of extreme blood loss and uncontrollable bleeding.

FVIIa is found only in very low concentrations in the plasma of healthy people. Very little is known to date about the formation and origin of the FVIIa circulating in the blood. It has therefore been assumed that traces of expressed thromboplastin or thromboplastin released during cell destruction might contribute to this. Despite intensive research into all the processes associated with coagulation, however, it has not to date been possible to find any evidence that constituents of the injured cell might make a crucial contribution to induction of hemostatic processes.

It has now been found, surprisingly, among the constituents released from injured tissues and cells the extracellular RNA represents an important initial cofactor for induction of the (a) extrinsic and (b) intrinsic coagulation cascade. This observation has prompted more detailed investigation of the function of the RNA or of bioactive fragments of the RNA, and of RNAses in hemostatic processes, and the development of pharmaceutical preparations to which natural or synthetic RNA or bioactive fragments of the RNA are added to promote hemostasis, or to which corresponding ribonucleases are added to inhibit the extracellular functions of RNA. Extracellular RNA represents in this connection the natural “foreign” surface for activating the plasma contact phase system, initiation of which has to date been described in vitro by kaolin or other polyanionic substances.

The invention therefore relates to a pharmaceutical preparation which comprises and amount, sufficient for promoting coagulation, of natural of synthetic RNA or of one or more coagulation-promoting fragments of RNA, peptide-nucleic acids, ribozymes or RNA aptamers. Such a preparation preferably additionally includes an activator for a plasma coagulation factor. A particularly suitable activator is (a) the factor VII-activating protease (FSAP) or its proenzyme and (b) components of the contact phase such as factor XII, kininogen and prekallikrein. The invention further relates to a pharmaceutical preparation of ribonucleases which abolish or prevent the procoagulant effect of RNA.

The pharmaceutical preparations of the invention are based on the realization that the RNA is the most effective cofactor for the proenzyme of FSAP and leads to activation of this enzyme. This interaction is achieved both for natural RNA from cell homogenates or supernatants of activated platelets and with fractions obtained from cytosolic RNA (especially ribosomal RNA), or synthetic RNA. There is evidently in this connection no cell-specific cofactor effect, because bacterial and viral RNA is also effective and the RNA molecules in the various cell types are very similar, so that apparently the high negative excess charge determines the functions of RNA as cofactor of FSAP.

The realizations are supported by the observation that FSAP is able to bind specifically to RNA and is also redissociated by a hypertonic saline solution. It is thus remarkable that, under physiological conditions, specific binding only by RNA, and not by DNA, to FSAP is detectable.

The invention is thus based on the realization that extrinsic activation of coagulation is induced by the interaction of RNA with FSAP, which is the most effective activator of the coagulation proenzyme factor VII. This coagulation pathway, which is specifically activated by tissue injuries, is thus set in train by the novel cofactor (natural or synthetic) RNA.

At the same time, RNA represents the natural cofactor for activation of the contact phase system, as has been shown in whole blood, plasma and in the purified system. This opens up novel and very diverse ways for exerting a positive or negative effect on the hemostatic processes through suitable pharmaceutical preparations.

A novel starting point for this is the use of RNA-degrading and inhibiting compounds for acting on hemostatic processes. If the coagulation-influencing cofactor RNA is inactivated by supplying RNA-cleaving or inhibiting substances, it is then no longer available for activation of FSAP or the contact system. It is evident from this that RNAses can abolish the effective FSAP and thus also render coagulation factor VII and the intrinsic coagulation ineffective. This leads to a new anticoagulant principle which can be utilized in therapy. RNA-degrading or masking components (such as, for example, binding proteins) can thus display important therapeutic effects which prevent highly specifically, without side effects, and selectively initiation of the coagulation system and thus have a pronounced anticoagulant or antithrombotic effect.

German Offenlegungsschrift 199 03 693 additionally discloses that factor VII-activating protease (FSAP) also has the property of bringing about efficient activation of single-chain urokinase (scuPA, single chain urokinase plasminogen activator) and single-chain tPA (sctPA, single chain tissue plasminogen activator), and is thus able to act as plasminogen activator activator (PAA). Activation of plasminogen activators can be determined in the presence of plasminogen in a coupled reaction also for the formation of plasmin itself or through the lysis, brought about by plasmin, of a fibrin clot.

It is evident from this that RNA or bioactive fragments of RNA also have a pronounced effect on the promotion of fibrinolysis.

The invention therefore further relates to pharmaceutical preparations which, in addition to an amount, sufficient to promote fibrinolysis, of RNA, one or more fibrinolysis-promoting fragments of RNA or RNA analogs such as peptide-nucleic acids, ribozymes or RNA aptamers, comprise an activator for a plasma fibrinolytic. It is possible and preferred to employ as activator for a plasma fibrinolytic the plasminogen activator-activating protease FSAP or its proenzyme. It is known in particular that FSAP is able to activate very efficiently the fibrinolytic properties of prourokinase. It is therefore possible also to use RNA for initiation and/or enhancement of fibrinolysis, through supporting the activation of the FSAP proenzyme, for example in the case of fibrin deposits or thromboses. It is possible in this connection to use RNA itself, its bioactive fragments or structures derived therefrom in the form of synthetically prepared molecules. Accordingly, RNA-degrading or inhibiting substances also have the effect of preventing the activation of FSAP and thus suppressing fibrinolysis.

The particular properties of FSAP have already led to the development of additional determination methods which are described in DE 199 03 693. The test method described therein can now be carried out considerably more specifically and accurately when the diagnostic aid also comprises a sufficient amount of RNA or of active fragments of RNA.

The invention therefore further relates to a diagnostic aid for the quantitative and qualitative detection of the coagulation factor VII activating protease FSAP or of its proenzyme, in which for determination
a) of the inactivating effect on coagulation factors VIII/VIIIa or V/Va or
b) of the shortening effect on coagulation times in global coagulation tests or
c) of the activating effect on plasminogen activators or
d) of the activating effect on factor VII
a sufficient amount of RNA or of bioactive fragments of RNA is employed.

The determination of inactivation of coagulation factors VIII/VIIIa or V/Va brought about by FSAP is based on incubating an FSAP-containing solution with the factor VIII/VIIIa or the factor V/Va and then measuring the remaining amount of said factor by means of a conventional activity assay and determining quantitatively the amount of FSAP therefrom by comparison with a standard plot. In this case, the protease activity is inhibited after preset periods by limited addition of aprotinin, which has the advantage that in these concentrations it does not influence the subsequent measurements of the assay system. The remaining activities of the coagulation factors are then measured by means of an assay familiar to the skilled worker. An assay system which has proved to be particularly appropriate for this employs the so-called Coamatic® factor VIII assay (Chromogenix AB) which essentially contains factors IXa and X, with the amount of FXa produced in the presence of a thrombin inhibitor being quantified by means of a conversion of a chromogenic substrate. This in turn is proportional to the amount of FVIII or FVIIIa.

Despite the FV and FVIII inactivation, it was possible to show that addition of FSAP to blood, to platelet-rich plasma or plasma shortened the coagulation times, that is to say the procoagulant effect was predominant in various so-called “global coagulation tests”. These test systems include for example the non-activated partial thromboplastin time (NAPTT), the prothrombin time (PT) and the recalcification time. Since the shortening of these coagulation times, measured for example in so-called coagulometers, by thromboelastography or else in chromogenic assays, correlates with the concentration of a procoagulant substance, it is possible conversely to draw conclusions about the concentration of the substance in a sample from the coagulation time on the basis of a calibration plot. Correspondingly, the concentration of FSAP can be determined with the aid of selected global coagulation tests.

The invention therefore further relates also to a diagnostic aid for the quantitative or qualitative detection of FSAP by determination of the effect shortening the coagulation times by means
a) of the non-activated partial thromboplastin time (NAPTT)
b) of the prothrombin time (PT)
c) of the plasma recalcification time or
d) of the activated partial thromboplastin time (APTT)
which comprises a sufficient amount of RNA or of bioactive fragments of RNA. The addition of RNA or its fragments also makes this test more specific and more sensitive.

The procoagulant activity of RNA can additionally be used to provide this cofactor in said diagnostic tests for determining coagulation times in whole blood or plasma. Besides natural RNA (transfer or ribosomal RNA), it is also possible to use synthetic RNA such as poly IC, poly C or poly AU as procoagulant cofactors, because they are more stable than natural RNA to ribonucleases (in blood, plasma). Said formulations are involved (independently of FSAP) in particular in the activation of the intrinsic contact phase and can therefore be employed in diagnosis for measuring corresponding coagulation times.

Finally, the activation, brought about by FSAP, in single-chain urokinase or single-chain tPA can also be used for a test system for detecting FSAP, which is further improved by the addition of RNA or of active fragments of RNA. In this case, the activity of activated plasminogen activators is measured with the aid for example of chromogenic substrates. The activation of the plasminogen activators can be determined in the presence of plasminogen in a coupled reaction also by the formation of plasmin itself or by a lysis, brought about by plasmin, of a fibrin clot.

The pharmaceutical preparations of the invention can be employed as coagulants either on their own or together with other substances which increase the protease activity, such as heparin or heparin-related substances such as heparan sulfate and/or calcium ions. The use of such a composition may for example be indicated with utilization of its FVIII inhibitor-bypassing activity (FEIBA) when there are intolerances of FVIII and/or FIX and/or FXI and/or the proteins of the contact phase, such as FXII, for example because of the presence of antibodies, or other types of deficiency situations are present. Use ex vivo of the pharmaceutical preparation of the invention for promoting coagulation is also possible for general prophylaxis of bleeding or for controlling bleeding.

On the other hand, activation of plasminogen activators by the pharmaceutical preparation of the invention can also bring about limited proteolysis of single-chain PAs, which is suitable for activation thereof. This leads to fibrinolysis through activation for example of prourokinase, which makes it appear that the use of the pharmaceutical preparations of the invention is indicated in thromboembolic disorders such as leg vein thrombosis, myocardial infarction or strokes.

The preparations of the invention can thus be used for the endogenous or exogenous activation of plasminogen activators such as prourokinase or tPA. This possible use is not contradictory to the fact that FSAP may also have procoagulant effects. The question of which of the two reactions predominates is probably controlled by the availability of the physiological substrates. According to the current state of knowledge, factor VII is moderately activated in plasma and permanently maintains a certain concentration of FVIIa in order to be able to counteract sudden vessel injuries immediately. By contrast, only ng amounts of tPA and urokinase plasminogen activator are present in 1 ml of plasma. Only in the event of fibrin deposition or thrombi is there an increase, through secretion or synthesis, in the concentration of plasminogen activators which, after activation, display their thrombolytic activity through plasminogen activation locally, in particular thrombus-bound. In the presence, especially locally restricted, of single-chain PAs, activation thereof might outweigh FVII activation, making adaptation to the physiological situation possible. Correspondingly, FSAP can also regulate hemostasis, making replacement of FSAP or of its proenzymes suitable for inborn or acquired deficiency states.

Since it has emerged that the plasminogen activator (especially prourokinase) enhancing effect of FSAP is promoted in particular by calcium and/or heparin or heparin-like substances such as dextran sulfate, it is possible for the lysis according to the invention of fibrin-containing thrombi to employ particularly advantageously pharmaceutical preparations which, apart from RNA or bioactive RNA fragments, additionally comprise soluble calcium salts and/or heparin or heparin-like substances. Further details of the invention are made clear in the following examples, without the intention to restrict the subject matter of the invention in any way thereby:

### EXAMPLE 1

### RNA from Various Sources Acts as Procoagulant Cofactor

RNA from
(a) cultured human fibroblasts (rRNA),
(b) yeast (tRNA),
(c) E. coli,
(d) Qβ phages (single-stranded RNA)
(e) synthetic source (poly IC, poly C, poly AU)
was employed in the same concentration without and after pretreatment with RNAse A in a turbidometric coagulation test, and the recalcification time was measured. All the RNA types were able to reduce the coagulation time significantly (quantification in kaolin equivalent units (KEU/ml)), while DNA shows no effect. Pretreatment of the RNA samples with RNAse A led to inactivation of the procoagulant activity; only in the case of E. coli RNA was this partially resistant to RNAse A treatment. This shows ambiguously that various types of polyanionic RNA have procoagulant activity; fragmentation of these RNA types into low molecular weight fragments leads to loss of the procoagulant activity. Compared with authentic rRNA or tRNA, synthetic single-stranded and double-stranded RNA showed procoagulant activity in coagulation tests in the following graduation: poly IC>poly C>poly AU>poly dIdC.

### EXAMPLE 2

### Behavior of RNA in the Plasma Milieu: RNA Memory

Although ribonucleases in blood (and in other body fluids) control the amount of circulating RNA, it was possible to determine the procoagulant function of RNA in plasma. In order to establish this connection, the fate of a defined amount of RNA in the plasma was followed over time: whereas high molecular weight RNA was degraded in plasma with a half-life of about 5 min and was no longer detectable after 30 min, the procoagulant activity of this RNA was still detectable unchanged after 90 min and longer. This phenomenon of the “RNA memory” is probably based on activation of RNA-dependent components in the plasma, which contribute to initiation of coagulation after recalcification. It is very probable that RNA in the plasma is thus also responsible for the phenomena of “latent” coagulation and of a “hypercoagulable” state, prediction or prevention of which is very important in the diagnosis and therapy of thrombotic complications.

### EXAMPLE 3

### Mechanism of RNA-Dependent Initiation of Coagulation

Whereas RNA added to plasma was able to increase the coagulation induced by tissue factor at every concentration of this cofactor, an equivalent effect of the RNA was evident in kaolin-induced coagulation. This indicates that RNA is involved as an anionic cofactor (foreign surface) in the initiation of the contact phase system of coagulation. In order to establish this fact, prekallikrein and kininogen were reacted in a purified system, and the amount of kallikrein formed was determined. In relation to the buffer control, the addition of RNA led to a significant increase in kallikrein formation, whereas pretreatment of the RNA with RNAse A prevented this effect. Experiments in factor XII- and factor VIII-deficient plasmas underline the function of RNA as contact phase cofactor, and in prekallikrein-deficient plasma (compared with normal plasma) RNA showed no effect. Taken together, these results proved that the natural material RNA, equivalent to kaolin (artificial material), sets the intrinsic initiation of coagulation in train after exposure with the contact phase system.

### EXAMPLE 4

### Detection of RNA in Patient's Plasma

Blood was taken in the presence of about 200 units/ml RNase inhibitor with subsequent preparation of the plasma or, alternatively, after taking of blood and obtaining of plasma stabilizer PAQ-gene (Qiagen) was added thereto in order to prevent RNA hydrolysis. After preparation of the RNA (via the Trizol method), it was immobilized on nylon filters, and radiolabeled rRNA-specific oligos were used to quantify after hybridization the amount of plasma RNA by means of a calibration plot. This analytic method was used to identify positive samples, whose RNA content was above that of healthy. donors, in plasmas from patients with acute myocardial infarction or sepsis.

### EXAMPLE 5

### RNA Antagonists

The procoagulant activity of RNA, especially in relation to the initiation of the plasma contact phase system, can be prevented by pretreatment of the RNA with RNAse A. It is thus plausible that an increase in the concentration of ribonucleases in the plasma leads to hydrolytic cleavage of the nucleic acid and thus ribonucleases display an anticoagulant effect. Since endothelial cells produce, and secrete into the blood, large amounts of RNAse A, as has been shown on cell cultures (Landre et al., 2002), an anticoagulant therapy with ribonucleases comes very close to the natural regulation principle in the vascular system. Alternatively, ribonucleases might also display an anticoagulant effect on ribozymes or RNA aptamers, because these substances might bring about, similar to natural RNA, contact phase activation.

### EXAMPLE 6

### Cellular Functions of RNA

Since RNA has a certain survival time also in the plasma milieu, the effect of polyanionic material on vessel wall cells, namely endothelial cell monolayers in culture, was tested. A very sensitive functional test for the cellular effect of extracellular RNA is the change in endothelial cell permeability, which was investigated on dense monolayers of microvascular cerebral pig endothelial cells. RNA increased in a concentration-dependent manner the permeability of these cells, and preincubation with RNAse was able to prevent this effect of extracellular RNA. An effect on endothelial cell permeability in the region of a vessel injury site is necessary for the onset of wound-healing responses, and thus great importance is attributed to this cellular function of extracellular RNA.

1. A pharmaceutical preparation which comprises an amount, sufficient for promoting coagulation, of natural or synthetic RNA or of one or more coagulation-promoting fragments of natural or synthetic RNA, RNA analogs, peptide-nucleic acids, ribozymes or RNA aptamers.

2. The pharmaceutical preparation as claimed in claim 1, which further comprises an activator for a plasma coagulation factor.

3. The pharmaceutical preparation as claimed in claim 2, which comprises factor VII activating protease or its proenzyme as activator.

4. A method for promoting coagulation, comprising administering the pharmaceutical preparation as claimed in any one of claims 1-3 to a patient.

5. A pharmaceutical preparation, which comprises an amount, sufficient for promoting fibrinolysis or inhibiting coagulation, of one or more RNA-degrading or -inhibiting compounds with ribonucleolytic activity or RNA-complexing capacity.

6. The pharmaceutical preparation as claimed in claim 5, which further comprises an activator for a plasma fibrinolytic.

7. The pharmaceutical preparation as claimed in claim 6, which comprises a factor VII-activating protease or its proenzyme as activator for a plasma fibrinolytic.

8. A diagnostic aid for detecting postoperative hypercoagulable states, complications of pregnancy, tumor status, acute myocardial infarction or sepsis, wherein said diagnostic aid is used in the detection of an increased plasma RNA content compared with healthy people.

9. A diagnostic aid for quantitative or qualitative detection of coagulation factor VII-activating protease or of its proenzyme, which comprises a sufficient amount of natural or synthetic RNA, of active fragments of natural or synthetic RNA or RNA analogs, peptide nucleic acids, ribozymes or RNA aptamers for determination
a) of the inactivating effect on coagulation factors VIII/VIIIa or V/Va or
b) of the shortening effect on coagulation times in global coagulation tests or
c) of the activating effect on plasminogen activators or
d) of the activating effect on FVII

10. A diagnostic aid as claimed in claim 8, which comprises a sufficient amount of natural or synthetic RNA, active fragments of natural or synthetic RNA, peptide-nucleic acids, ribozymes or RNA aptamers for determination of the effect shortening the coagulation time by means
a) of the non-activated partial thromboplastin time (NAPTT) or
b) of the prothrombin time (PT) or
c) of the plasma recalcification time or
d) of the activated partial thromboplastin time (APTT)

11. A diagnostic aid as claimed in claim 8 or 9, which comprises a sufficient amount of natural or synthetic RNA, active fragments of natural or synthetic RNA, peptide-nucleic acids, ribozymes or RNA aptamers for determination of the effect activating or enhancing the plasminogen activators through the activation
a) of single-chain urokinase (scuPA, single-chain urokinase plasminogen activator) or
b) of single-chain tPA (sctPA, single-chain tissue plasminogen activator)

12. A pharmaceutical preparation as claimed in claim 5, which comprises an amount, sufficient for the treatment of sepsis, of one or more RNA-degrading, inhibiting or complexing compounds.
